(19) United States
(12) Patent Application Publication
(10) Pub. No.: US 2011/0224104 A1
(43) Pub. Date: Sep. 15, 2011

(54) METHOD AND SYSTEM FOR INDENTIFICATION OF MICROORGANISMS

(75) Inventors: Evgenia Razumovski, Columbia, MD (US); Berk Oktem, Columbia, MD (US); Appavu K. Sundaram, Montgomery Village, MD (US); Vsevolod S. Rakov, New Haven, CT (US); Sudeepta Shanbhag, Ellicott City, MD (US); Sergey Kurnosenko, Fairfax, VA (US); Vladimir M. Doroshenko, Ellicott City, MD (US)

(73) Assignee: Science & Engineering Services, Inc., Columbia, MD (US)

(21) Appl. No.: 11/757,020

(22) Filed: Jun. 1, 2007

Related U.S. Application Data
(60) Provisional application No. 60/911,757, filed on Apr. 13, 2007.

(51) Int. Cl. C40B 50/06; C12Q 1/04; C12M 1/34; C40B 60/14

(52) U.S. Cl. 506/26; 435/34; 435/287.1; 506/40

(57) ABSTRACT

A method and system for identification of microorganisms in a sample. The method includes processing the sample to produce at least one group of biomarker molecules from at least one microorganism in the sample, tandem mass-analyzing biomarker fragment ions from the at least one group of biomarker molecules to obtain a sample biomarker tandem mass spectrum of the biomarker, and identifying the microorganism in the sample based on a comparison of the sample biomarker tandem mass spectrum to an experimentally-derived reference tandem mass spectrum (stored in the reference library) from a known microorganism. The system includes a sample processing unit configured to process the sample and produce at least one group of biomarker molecules from at least one microorganism in the sample. The system includes a mass-analyzer for tandem mass analysis biomarker fragment ions. The system includes a processor configured to identify the microorganism in the sample based on a comparison of a sample biomarker tandem mass spectrum to an experimentally-derived reference tandem mass spectrum from a known microorganism.

### CROSS REFERENCE TO RELATED APPLICATIONS

This application is related to and claims priority under 35 U.S.C. §1119(e) to U.S. Ser. No. 60/911,757 entitled “METHOD AND SYSTEM FOR IDENTIFICATION OF MICROORGANISMS” filed Apr. 13, 2007, the entire contents of which are incorporated herein by reference. This application is related to U.S. Ser. No. 11/441,176 entitled “METHOD AND APPARATUS FOR PROCESSING OF BIOLOGICAL SAMPLES FOR MASS SPECTROMETRY ANALYSIS” filed May 26, 2006, the entire contents of which are incorporated herein by reference. This patent application is related to U.S. Ser. No. 11/144,666 entitled “METHOD AND APPARATUS FOR IONIZATION VIA INTERACTION WITH METASTABLE SPECIES” filed Jun. 6, 2005, the entire contents of which are incorporated herein by reference. This patent application is related to U.S. Ser. No. 11/126,215 entitled “METHOD OF ION FRAGMENTATION IN A TANDEM MASS SPECTROMETER” filed May 11, 2005, Attorney Docket No. 271841US41, the entire contents of which are incorporated herein by reference.

### BACKGROUND OF THE INVENTION

1. Field of the Invention

The present invention relates to rapid detection of microorganisms in environmental and biological samples using mass spectrometry.

2. Discussion of the Background

Rapid detection of multiple microorganisms in complex biological samples is becoming critically important in various areas, such as medical diagnostics, detecting public health hazards and monitoring potential biological terrorism. Events such as anthrax-tainted mail and acts of terrorism have elevated the demand for suitable instrumentation to detect and identify potentially threatening biological agents, such as bacteria, viruses, toxins and chemical agents in complex mixtures. This heightened demand for methods to rapidly and concurrently identify possible threats exceeds current technology.

There are a few methods currently used for organism detection in complex biological mixtures. All these methods target specific components of a bio-agent of interest to unambiguously detect its presence in the mixture of other organisms. One such methodology, referred to as polymerase chain reaction (PCR), methodology is based on the concept of DNA hybridization—a set of oligonucleotide primers for a particular organism is added to the DNA sample. If the primers are complementary to the DNA in the sample, they hybridize, are amplified, and finally the organism is detected. If there is no hybridization, it is assumed that the organism's DNA is not present in the sample.

There are a number of disadvantages to the PCR approach. For example, 1) DNA can be altered through genetic manipulations while it is significantly more difficult to alter proteins. 2) An unique template DNA from the organism in the sample is needed in order to detect any new organisms. 3) All the components of PCR (such as primers) must be made stable over long periods of time.

Another method for organism detection in complex biological mixtures is referred to as antibody based detection method. In this method, antibodies are developed to recognize organism specific proteins. When the proteins are present in the sample, the antibodies will bind to them producing a detection response.

There are a number of disadvantages to the antibody based detection approach. For example, 1) the development of new antibodies can be difficult and time consuming. This constraint, therefore, limits its usefulness for the detection of new organisms. 2) It can be difficult to select a combination of proteins which will be specific to only one specific organism strain. 3) The antibodies may not be specific to an organism in the truly complex background. When more organisms are introduced into the sample the chances of another organism possessing the protein that can non-specifically bind to the designed antibody increases.

Mass spectrometry: Mass spectrometry (MS) is known to be a fast and reliable analytical technique for measuring masses of biological molecules in complex mixtures (See Pandey et al., Nature, 2000, 405, 837-846, the entire contents of which are incorporated herein by reference). Using mass analysis, it is possible to characterize proteomic sample composition. Fundamentally, a mass spectrometer is an instrument including three parts: an ionization source, an ion analyzer and an ion detector. The ionization source is responsible for desorption and ionization of biological molecules into a gas phase, the ion analyzer separates the gas molecules according to their mass to charge ratios (m/z) and the ion detector detects and multiplies the ion signal. The result of MS analysis is a two-dimensional spectrum with m/z and intensity axes, where intensity is a function of the number of molecules of the same m/z present in the sample. Two ionization sources frequently used in proteomic analysis are electrospray (ESI) and matrix assisted laser desorption ionization (MALDI) sources.

The MALDI ionization technique involves transferring biological molecules into a gas phase from a solid matrix (Hillenkamp et al., Anal Chem., 1991 Dec. 15; 63(24):1193A-1203A, the entire contents of which are incorporated herein by reference). Some of the different MALDI ionization techniques involve atmospheric pressure (AP) MALDI, and the use of infrared or ultraviolet lasers for the ionization. The MALDI ionization method produces low charge state molecules—the ions measured by a mass analyzer have in most cases single, double and, more rarely, triple charges. Electrospray Ionization (ESI) transfers biological molecules directly from liquid matrix into gas phase (Fenn et al., Science, 1989, 246, 64-71, the entire contents of which are incorporated herein by reference).

In the process of producing mainly multiply-charged molecular ions, either of these ionization sources can be coupled with a variety of mass analyzers such as sector MS, quadrupole mass filter, quadrupole ion trap (QIT), linear ion trap, time-of-flight (TOF), Orbitrap, ion cyclotron resonance (ICR, FTICR, FTMS) mass spectrometers, and any of their tandem-in-space hybrids.

The masses of biological molecules reflect their composition and are used for their detection and characterization. One described MS method for microorganism identification, referred to as a spectral fingerprint technique (See U.S. Pat. No. 6,177,266 the entire contents of which are incorporated herein by reference), is based on measuring the m/zs of proteins and other biomolecules originated from an organism membrane or inside the cell. It involves collecting MS signatures of various microorganisms and detecting the presence of organism-specific m/z peaks on the MS level. This method can be used to detect several bacteria: Bacillus anthracis, Bacillus thuringiensis, Bacillus cereus, Bacillus subtilis, Yersinia pestis, Francisella tulansis and Bucella melitensis.

The success of this method is dependent upon being able to detect a specific signature of a particular organism in MS spectrum, which becomes increasingly more difficult with the increased complexity of a biological sample as well as significantly decreased limit of detection (as the amount of clutter in the sample increases, the biomarker peaks become buried and finally undetectable). This method has been shown to work well with pure samples but it is generally less applicable to the microorganism mixtures. It is also hampered by background interferences and changes in microorganism growth conditions.

Tandem MS: Tandem MS experiment involves the following three steps: isolation of ions in a particular mass to charge ratio, fragmentation of these ions, and the detection of the resulting fragment ions. The fragmentation is frequently performed by a process called collision induced dissociation (CID), which involves colliding peptides of isolated m/z with inert gas, the collisions inducing breaks in the peptide bonds, and resulting in a spectrum of mass to charge ratios for peptide fragments, which are the function of the peptide sequence. There are also other dissociation methods available today, that provide peptide ion fragmentation information, such as electron transfer dissociation (ETD) (See Syka et al., PNAS, 2004, 101(26): 9528-9533, the entire contents of which are incorporated herein by reference), electron capture dissociation (ECD) (See Zubarev et al., J. Am. Chem. Soc.1998,120,3265-3266, the entire contents of which are incorporated herein by reference), and electron detachment dissociation (EDD) (Budnik et al., Chem. Phys. Letters, 2001, 342,299-302, the entire contents of which are incorporated herein by reference), multiphoton dissociation and fragmentation using interactions with metastable molecules.

Using the precursor m/z of the peptide and its fragmentation pattern, the identity of a biological molecule can be established through a variety of database search algorithms. The use of mass spectrometry for high-throughput protein detection in a one organism mixture (See Aebersold et al., Nature, 2003, 422(6928):198-207, the entire contents of which are incorporated herein by reference), presents an approach to characterize proteomic content of multiple organisms, and using the species-specific sequences it is possible to detect the presence of a particular species in a complex biological sample.

Low energy fragmentation: During low energy fragmentation, isolated peptides fragment break along peptide backbone bonds, each break creating a pair of fragment ions. The fragment ions retaining N-terminus of a peptide are referred to as ‘a’, ‘b’, ‘c’ ions, while the ions containing C-terminus are referred to as ‘x’, ‘y’, ‘z’ ions (See Biemann K., Biomed. Env. Mass Spec., 1988, 16, 99, the entire contents of which are incorporated herein by reference). Historically, the first and still most commonly used induced fragmentation method is the so-called collision-induced dissociation (CID) technique which employs collisions of fast-moving ions with relatively slow molecules of low-vacuum background gas (air, nitrogen, argon, helium, etc) yielding mostly “y” and “b” type peptide-fragment ions. Similar fragmentation patterns are observed in a variety of multiple-photon induced dissociation MS/MS methods, while the spectra dissociated using ETD, ECD and EDD are dominated by “c” and “z” type ions.

CID fragmentation: In the case of CID fragmentation the expected outcome is a pair of ‘b’ and corresponding ‘y’ ions appearing at each amino acid position and producing so called ion series in MS/MS spectrum. The m/z difference between the two singly charged consecutive ions of the same type (either two ‘b’ or two ‘y’) are equal to a single amino acid mass and are used to infer the identity of the amino acid. This property is extensively used by many of the peptide identification algorithms to infer the sequence information from the tandem MS spectrum. While ‘b’ and ‘y’ ions are the major ions in the spectrum, other types of ions may be present, but are expected to appear at a significantly lower abundance. Each ion appears in the tandem MS spectrum in the form of a peak with an m/z and intensity. Intensity of a peak is related to the abundance of the ions of this m/z.

In theory, the number of ‘b’ and ‘y’ ions for each given peptide is roughly equal to the number of the peptide bonds. If the number of amino acids in the peptide is equal to N, then the peptide will have a maximum of N-1 ‘b’ and N-1 ‘y’ ions. However, it has long been known that tandem MS spectra generally contain significantly more peaks, while not all the expected ones are observed. The chemistry behind the peptide fragmentation process is complex. The peak intensity values, while repeatable under the same instrumental conditions, are considerably different for different peptides, depending on the peptide amino acid sequence (See Wysocki, et al., J. Mass Spectrom. 2000, 35, 1399-1406, the entire contents of which are incorporated herein by reference). Other peaks besides the expected ‘b’ and ‘y’ ions may appear. The discrepancy in fragment peak intensities is especially pronounced for low charged peptides, like the singly-charged peptide ion produced in MALDI. The peak intensity is a valuable source of peptide sequence information which has not yet been successfully utilized by the majority of data interpretation applications.

Bottom Up Proteomics: Bottom up is a term used for one of the main methods for high-throughput proteomic experiments, for which instrumentation is well developed, robust and routine to operate. A typical procedure of complex mixture characterization using a bottom up approach involves a protein mixture digestion with an enzyme protease (such as trypsin, pepsin, glu-C, etc) followed by MS and MS/MS analysis. MS analysis can be performed with either ESI or MALDI ionization techniques coupled with a mass analyzer. The MS analysis yields information about the mass to charge ratio of the examined peptide while MS/MS analysis produces a fragmentation spectrum providing information about the peptide amino acid sequence. A variety of data interpretation techniques, where the main method is currently database search (DB search), can then be used to identify and detect the peptide, the protein and the organism from which the peptide might have originated (Pribil et al., 2005, J. Mass Spectrom., 40, 464-474, the entire contents of which are incorporated herein by reference). U.S. Pat. No. 7,027,933, the entire contents of which are incorporated herein by reference, describes a classification process that can be used for mass spectra.

Database Search: Bottom up proteomics heavily relies on database search engines in order to interpret experimental mass spectral data. There are many available proteome database search algorithms for MS data interpretation, such as MASCOT, SEQUEST, DBDigger, Sonar, ProteinProspector, and OMSSA. U.S. Pat. No. 6,940,065, the entire contents of which are incorporated herein by reference, describes a search process that can be used for mass spectra including a discussion of MASCOT and other search routines. Database search algorithms rely on a comparison between the theoretical fragmentation patterns of the database derived peptides and the experimentally observed fragmentation pattern. First, these search algorithms select a list of candidate database peptides, producing theoretical fragmentation patterns for each of them, and then compare the theoretical to an experimentally measured tandem MS spectrum. The theoretical peptide whose spectrum displays the highest spectrum similarity to the experimental spectrum is accepted as the best candidate and can be reported as identification.

There are typically two basic assumptions that can be met in order for an identification to be successful. The first assumption is that the peptide represented by the tandem MS spectrum is present in the database. The second assumption is that the correct peptide's theoretical spectrum is more similar to its experimental tandem MS spectrum than the theoretical spectrum of any other peptide in the database. The database identifications involving the discussed comparison between theoretical and experimental MS/MS spectra are frequently not accurate due to the absence of intensity and preferential fragmentation information in the theoretical spectrum.

While database search remains the method of choice for peptide detection (for reasons of availability, correctness of general principles, and unified approach), it frequently lacks the necessary precision for accurate organism identification in a complex biological sample. Additionally, database searches are only available for particular types of fragmentation methods, for which the theoretical fragmentation principles are available (like “y” and “b” ion series). Experimentally observed peptide fragmentation patterns are dependent on both instrument and experimental conditions. For some dissociation methods, theoretical fragmentation rules are poorly developed, and accurate theoretical spectrum cannot be constructed. When the theoretical and experimental spectra are not comparable, database search algorithms do not work. At the same time, since fragmentation pattern of a peptide is repeatable if fragmented under similar conditions, experimental MS/MS spectra measured under similar conditions can be compared without theoretical knowledge of fragmentation rules.

### SUMMARY OF THE INVENTION

In one embodiment of the present invention, there is provided a method for identification of microorganisms in a sample. The method includes processing the sample to produce at least one group of biomarker molecules from at least one microorganism in the sample, tandem mass-analyzing biomarker fragment ions from the at least one group of biomarker molecules to obtain a sample biomarker tandem mass spectrum of the biomarker molecule, and identifying the microorganism in the sample based on a comparison of the sample biomarker tandem mass spectrum to an experimentally-derived reference tandem mass spectrum from a known microorganism.

In one embodiment of the present invention, there is provided a system for identification of microorganisms in a sample. The system includes a sample processing unit configured to process the sample and produce at least one group of biomarker molecules from at least one microorganism in the sample. The system includes a mass-analyzer configured to receive the at least one group of biomarker molecules from the sample processing unit and to tandem mass analyze biomarker fragment ions from the at least one group of biomarker molecules to obtain a sample biomarker tandem mass spectrum of the biomarker molecule. The system includes a processor configured to identify the microorganism in the sample based on a comparison of the sample biomarker tandem mass spectrum to an experimentally-derived reference tandem mass spectrum from a known microorganism.

In one embodiment of the present invention, there is provided a computer program element encoded in a memory of a processor to identify the microorganism in the sample based on a comparison of the sample biomarker tandem mass spectrum to an experimentally-derived reference tandem mass spectrum from a known microorganism.

It is to be understood that both the foregoing general description of the invention and the following detailed description are exemplary, but are not restrictive of the invention.

### BRIEF DESCRIPTION OF THE DRAWINGS

A more complete appreciation of the present invention and many attendant advantages thereof will be readily obtained as the same becomes better understood by reference to the following detailed description when considered in connection with the accompanying drawings, wherein:

FIG. 1 is a schematic illustrating one embodiment of the invention for multiple microorganism detection using MS/MS typing;

FIGS. 2A-2C are a schematics illustrating the differences between the information content of experimental MS/MS spectra and a theoretical spectrum for the same peptide;

FIGS. 3A and 3B are schematics illustrating the receiver operating characteristic (ROC) curves of a MASCOT database server performance vs the MS/MS typing performance according to one embodiment of the present invention;

FIG. 4 is a schematic illustrating the rate of false positive identifications versus rate of true positive identifications as a function of a Pearson correlation coefficient;

FIG. 5 is an AP-MALDI-MS/MS spectrum of peptide LVSFAQQNMSGQQF, with precursor mass of m/z 1584.74;

FIG. 6 is a schematic illustrating the process of identifying Bacillus globigii using MS/MS typing according to one embodiment of the present invention;

FIG. 7 is a flowchart depicting a method according to one embodiment of the present invention;

FIG. 8 is a schematic depicting a system implementation according to one embodiment of the present invention.

### DETAILED DESCRIPTION OF THE EMBODIMENTS

MS/MS typing is a method to rapidly detect multiple microorganisms in a complex biological sample. The observation and detection of microorganisms is performed by measuring and analyzing their unique species-specific peptide biomarkers by mass spectrometry. The biomarkers yield tandem MS signatures of unique species specific peptides, experimentally measured and recorded into a Reference Library together with the corresponding biomarker precursor masses.

A spectral fingerprint method historically was one of the first MS-based method used for microorganism identification. However, due to a number of shortcomings, it was not especially suitable for real environmental samples. MS-based methods for microorganism detection typically rely on identifications by means of database search. In general, the development of MS methods for microorganism identification resembles those for identification of proteins and peptides, discussed above. With MS, a sample containing a microorganism can be chemically processed to select a group of peptides whose masses can be measured. Only the peptides that are always detectable in the presence of a particular organism and that are not detectable in its absence can be used for organism identification. These peptides, which are unique to one species, are referred to as “biomarker peptides” and become the microorganism's signature peptides (i.e., the peptides used to detect a corresponding organism).

Thus, it is possible to infer the presence of a specific microorganism in a complex sample by monitoring the presence of its biomarker peptides. It is possible to observe and identify biomarker peptides by bottom up (MS/MS) proteomics techniques. While the method can work with mixtures of microorganisms and in the presence of environmental and biological clutters, its sensitivity is limited by the lack of an adequate understanding of fragmentation rules, such as preference for the long peptide ion fragment series and the requirement for high spectral quality, frequently related to the concentration of the microorganism in the sample. The present invention in various embodiments provides novel methods which are sensitive enough for samples with limited amounts of microorganisms and have high detection reliability for biomarkers with incomplete fragmentation ion series.

According to one embodiment of the present invention, MS/MS typing is used as a bottom up method for microorganism detection based on detection of a MS/MS signature of its species-specific biomarker peptide. MS/MS typing utilizes a direct comparison of a measured spectrum with a reference MS/MS spectrum (usually obtained in a separate experiment) contained in the reference library, via spectral correlation techniques. This method is applied to proteomics tasks for various peptide identifications (so called targeted proteomics, for example the MS/MS library compiled by NIST) but has not heretofore been applied for microorganism identification.

The advantages of microorganism identification by the MS/MS typing method of the present invention are: the ease of including fragment ion intensities in the peptide biomarker identification process; high speed of identification; better sensitivity due to the presence of peak intensities; the ability to use MS/MS instruments with moderate mass resolution and accuracy (which are required for database search); insensitivity to background and growth conditions; and applicability to analyzing microorganism mixtures (due to MS/MS—in contrast to MS method for organism identification).

The ease of including fragment ion intensities in the peptide biomarker identification process is, in one embodiment, due to the presence of a reference library biomarker spectrum whose intensities are expected to be linearly correlated to the biomarker spectrum measured during the process of organism detection. The degree of the correlation can be measured with such methods as correlation coefficient. An additional benefit is that as long as the fragmentation is expected to be repeatable (e.g., occurs under similar fragmentation conditions, the same sequence will always fragment in the similar manner, and a appropriate fragmentation spectrum is present in the reference library) the fragmentation techniques used in different embodiments of the present invention do not affect the comparison to be made, while during traditional database search new theoretical models of fragmentation may need to be established.

Accordingly, the present invention in many embodiments permits a higher speed identification of a specific organism than conventional analysis involving selecting high precursor ions for fragmentation and identifying the precursor ions with database search algorithms. The higher speed is due to a) reduced time in mass analysis as only a single (or a few) MS/MS spectra on specific precursor masses for that organism's biomarker peptides are required rather than all of the present precursor ions and b) reduced time for data interpretation as only one (or a few, based on the number of obtained MS/MS spectra) comparison has to be made rather than comparisons against full sequence database (which frequently includes thousands possible peptides-theoretical spectra). These benefits are beyond that which would be possible with the conventional bottom up proteomic approach for organism detection.

Moreover, the present invention in many embodiments is less sensitive to background and growth conditions and more applicable to analyzing microorganism mixtures due to performing a MS/MS fragmentation rather than looking at MS spectrum. A conventional MS spectrum reflects the presence of all peptide ions present in the sample, in order to detect an organism. In other words, the peptide ions of the organism must be visible above all the other ions (i.e., must be present at higher concentration than the background clutter), which is frequently an unrealistic assumption. However, in case of MS/MS fragmentation, the desired precursor mass is isolated and fragmented, the other ions are excluded from the analysis, thus, background noise does not affect the detection as much in the present invention and the concentration of the organism of interest in the sample does not need to exceed the concentration of clutter.

Despite the complexity of MS spectrum which is greatly influenced by the amount of clutter, tandem MS spectra are influenced by the clutter to a significantly lesser degree, due to the effect of observing only a selected mass range of the spectrum, while discarding the other (including background) ions. This results in an amplification of the signal in that range to the necessary level for peptide identification.

The present invention in one embodiment introduces a methodology to replace database search analysis for peptide and organism identification and detection, increasing the speed and accuracy of the analysis. The present invention in one embodiment utilizes spectral comparison techniques, similar to the ones involved in database searches, but applied to a comparison between two experimental spectra, without the loss of intensity information. This methodology uses a reference library containing experimental MS/MS (i.e., tandem) spectra of species-unique biomarkers for bio-agent detection.

Accordingly, the time to analyze the experimental MS/MS spectrum for bio-agent detection is significantly reduced because only one or a few spectral comparisons (determined by the number of entries in the reference library sharing the same precursor m/z with the observed experimental spectrum) are necessary instead of thousands made by conventional database searches. The average time to perform an MS/MS search using MASCOT on a database containing 3888187 sequences on a processor Xeon 3.20 GHz computer takes on the order of 1 minute. While the processor speeds are expected to increase, so do the database sizes. A single correlation comparison between two spectra takes less than a second, a time which is not expected to increase since only one comparison is needed for one MS/MS verification.

The use of a reference library MS/MS spectra increases the sensitivity and the specificity of identifications. The presence of intensity information in the experimental database reference library allows more definite identifications especially in the case of peptides with only limited fragmentation capacity (few fragment peaks, rather than complete ‘b’ and ‘y’ series), which frequently appear in MALDI MS/MS spectra.

In other words, the experimental database reference library spectra contain full intensity information, including peptide fragmentation specifics. The absence of some of expected ‘b’ and ‘y’ ions, as well as the presence of atypical ions in the spectrum, the feature of which often make it difficult for database searches from theoretical spectra to produce a good identification. However, under the same conditions, the peptide retains the majority of its fragmentation features, and two experimental MS/MS spectra of the same peptide are significantly more similar to each other than to a theoretical spectrum not containing peak intensity information.

Additionally, the use of experimental database reference libraries allows for fast and comparably easy transitioning between different ion dissociation methodologies, by simply performing experiments with new type of dissociation method (for example ECD) on a precursor ion, and by adding the newly obtained reference MS/MS spectrum into the experimental database reference library.

As detailed below, the present invention in one embodiment can provide an improved method for detection of specific bio-agents in complex mixtures using MS/MS typing. The method can be applied to detection of any organism which contains an MS/MS measurable biomarker peptide.

As detailed below, the present invention in one embodiment can create procedures for composing a library containing MS/MS biomarkers for the bio-agents of interest and provides in one embodiment a method for detecting bio-agents using the compiled MS/MS library.

As detailed below, the present invention in one embodiment can increase the sensitivity and reliability of bio-agent detection and identification by including ion peak intensity information into the process of matching between reference and measured MS/MS spectra.

As detailed below, the present invention in one embodiment can use spectral correlation methods for scoring the match between experimental reference and measured MS/MS spectra.

As detailed below, the present invention in one embodiment can provide a faster response method for bio-agent detection and identification via the use of MS/MS instruments than the conventional techniques described above.

With reference to the figures, the present invention includes a method, system, and computer program product (for example containing programming instructions) to detect bio-agents in complex samples using mass spectrometry MS/MS typing. The following processes can be utilized: A reference library for MS/MS typing can be created, or may have already have been created. A sample is processed to prepare the sample for MS analysis. The sample is mass analyzed using MS and/or MS/MS analysis. Microorganism identification is performed using MS/MS typing.

Creating Reference library: Creating an experimental reference library involves storing masses and MS/MS spectra of species-unique biomarkers which will later be used for MS/MS typing. The reference library is designed to contain a representation of the microorganisms in a form of a single or multiple MS/MS spectra unique to each organism, along with their corresponding biomarker precursor masses.

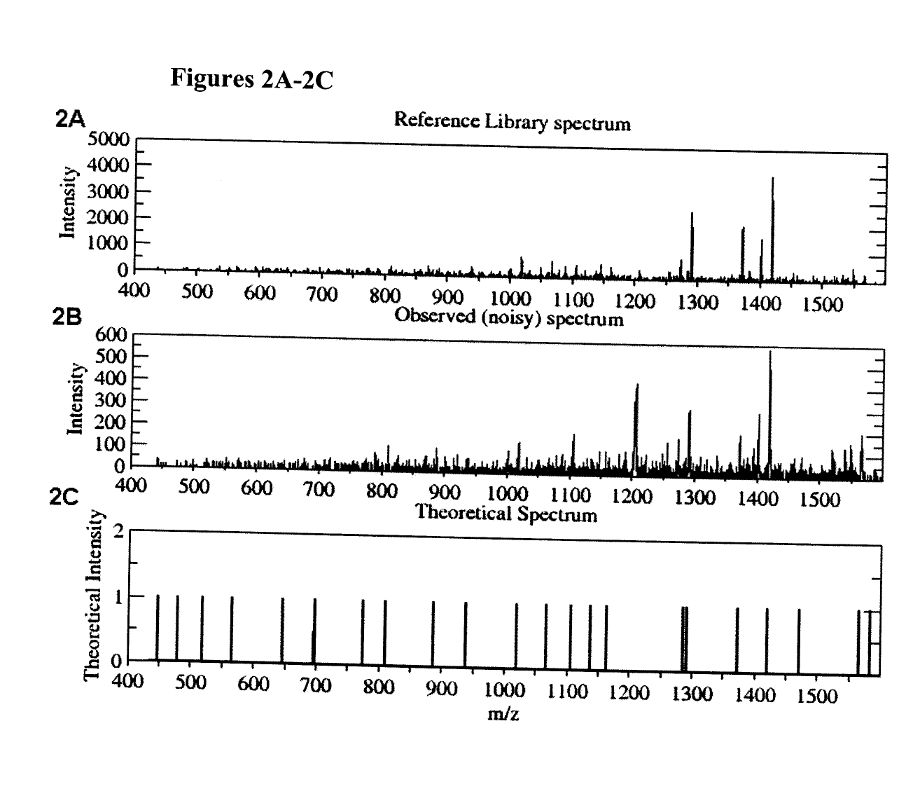

Once a peptide's experimentally measured fragmentation spectrum is available, it is significantly more representative of future fragmentation patterns as compared to a theoretically predicted MS/MS. FIG. 2 shows that a reference library spectrum more closely matches a typical noisy measured mass spectrum than a theoretical mass spectrum. While there are many types of fragmentation methods based on different physical principles, all (or most) of the fragmentation methods are applicable to the present invention's methodology, because recording a spectrum is not dependent on the fragmentation method as database search theoretical spectra predicators. As noted above, a reference library MS/MS spectrum and a noisy measured MS/MS spectrum are significantly more similar to each other than to a theoretical MS/MS spectrum having invariable intensities for all predicted ions.

Creating the experimental reference library can involve but is not limited to: a) in-silico determining of unique peptide biomarkers that can be used to represent specific microorganism through the use of known computer program selection; b) experimentally measuring the MS/MS spectra of the biomarkers and including the experimental MS/MS reference spectrum in the reference library to represent the organism; c) in case it is not possible to experimentally measure microorganism MS/MS spectrum, a theoretical reference MS/MS spectrum can be added to the reference library, preferably, with predicted ion fragment intensities (one example of such predictors is published by Elias et al., Nat Biotechnol. 2004 February; 22(2):214-9, the entire contents of which are incorporated herein by reference).

In order to in-silico determine species-unique peptide biomarkers for microorganisms of interest, the amino acid sequences for the potential target proteins are derived computationally. The sequences are subjected to in-silico proteolysis (corresponding to the experimental proteolytic procedure described below) to create theoretical peptides. Then, each of the peptides is compared to the complete list of known sequences present in all sequenced organisms to verify its uniqueness to the targeted bio-agent, producing the initial list of potential peptide biomarkers. If the organism is not fully sequenced, it is also possible to directly provide several amino acid sequences from the microorganism, whose uniqueness can be verified using a similar strategy.

The term “uniqueness” used throughout this document refers to the area of applicability of certain methods, systems, and products of the invention. The biomarkers can be unique enough to identify the bio-agent in typical environment(s) where this agent can be found or placed. For example, for environmental samples the biomarker peptide can not be expressed as a result of processing and MS/MS analysis of similar environmental samples not containing the bio-agent. This means that the peptide sequence can be unique for all organisms that could be present in this environmental sample and expressed in the spectra after sample processing and analysis (since not all sample peptides are equally expressed in the MS spectra).

Once the in-silico biomarker peptides have been established, their MS/MS spectra are obtained using mass spectrometry to create experimental reference MS/MS spectra. This process involves a) subjecting isolated and concentrated microorganisms to appropriate sample processing and mass analyzing procedure (to be described below) which will later be used for the organism detection, b) collecting precursor peptide ion information from MS analyzer and c) MS/MS fragmenting the precursors which correspond to the in-silico biomarkers to receive corresponding MS/MS spectra. Then, the relationship between collected MS/MS spectra to the biomarker sequence is verified (which can be determined for example by de novo sequencing, database search or simple peak to peak comparison) and saving the verified MS/MS spectra into reference library as species-unique reference spectra for the microorganism.

In case the microorganism is not available for experimental analysis, theoretical MS/MS spectra can be created to represent this microorganism in the reference library. In order to create theoretical spectra, peptides are subjected to in-silico MS/MS fragmentation, which produces a sequence specific series of ‘b’ and ‘y’ ions (in case of OD fragmentations, and other patterns in case of a different type of dissociation), with intensities calculated using intensity predicting programs to model real ion fragment intensities.

Sample processing: Sample processing can, but is not limited to, involving at least one of following steps: a) concentrating a sample to increase amount of analyte per unit volume; b) solubilizing a selected group of proteins by subjecting the sample to a solution containing an appropriate solvent, such as acid, base or an organic solvent; c) digesting the sample proteins into smaller peptides via proteolysis, typically using trypsin; d) cleaning up the sample to remove salts, buffers and other impurities before mass spectrometry analysis (for example using C18 ZipTips). More sample processing methods are described in Application for U.S. patent Ser. No. 11/441,176, the entire contents of which are incorporated herein by reference.

Mass analyzing: Mass analyzing can, but is not limited to, involve the following steps: a) ionizing the sample, b) MS analyzing the sample, c) acquiring MS/MS of specific precursor m/z ranges corresponding to the biomarkers present in the reference library (for example using AP-MALDI ion source interfaced to a QIT mass spectrometer).

Ionizing the sample can be performed using any of the available ionization techniques, including, but not limited to, ESI and MALDI. MS analyzing of the sample is performed in order to detect potential fragmentation targets which are found by cross-referencing the peptide precursor masses present in reference library and precursor masses in just-observed MS spectrum. MS/MS can either be performed directly on a particular m/z range corresponding to a specific reference library biomarker without prior MS analysis, or on a series of selected m/z ranges corresponding to the potential target species detected in MS analysis. MS and MS/MS analysis can be performed using any available mass analyzers, including but not limited to QIT, Q-TOF, FTICR-MS.

Microorganism identification: Microorganism identification in one embodiment of the present invention uses tandem mass spectroscopy. For example, microorganism identification using MS/MS typing involves a) searching for a match between the MS/MS spectrum observed from the sample and a spectral biomarker MS/MS spectra in the reference library using, for example, spectral correlation techniques, and b) detecting the presence of the bio-agent based on the result of the correlation analysis.

Mass spectroscopy for example as MS/MS typing is based upon comparison of two MS/MS spectra to determine whether the spectra originate from the same peptide. Under comparable data collecting conditions, similar fragmentation patterns are obtained for similar peptides.

MS/MS comparisons can be accomplished using spectral correlation analysis instead of standard database search. This analysis determines correlation coefficient, a measure of the strength and direction of the linear relationship between two random variables. In one embodiment of the present invention, such correlation analysis is applied to establishing the relationship between two MS/MS spectra: the bio-agent representative MS/MS spectrum contained in the reference library, and the MS/MS spectrum measured in the given sample. If the strength of relationship between the experimental MS/MS and the reference library, based on the correlation coefficient, is deemed significant, and the presence of corresponding bio-agent in the analyzed sample is then reported as detected with a certain confidence (or significance) derived from the value of the correlation coefficient.

There are several correlation methods with similar discrimination properties for comparisons between two mass spectra. Each of these methods can be applied to the methodology of the present invention. Some of the possible spectral correlation methods are: Pearson Correlation/Cosine similarity measure, Spearman Correlation, cross-correlation, regression analysis, χ²-method, etc. In the following examples, Pearson correlation is used.

The standard approach to compare efficiency of data analysis methods is building a receiver operating characteristic (ROC) curve, which involves plotting sensitivity and 1-specificity of the methods, where sensitivity is defined as

$$\frac{TruePositiveIdentifications}{TruePositiveIdentification + FalseNegativeIdentifications}$$

and 1-specificity is defined as

$$\frac{FalsePositiveIdentifications}{FalsePositiveIdentifications + TrueNegativeIdentifications}$$

The sensitivity corresponds to the fraction of True Positive (TP) Identifications out of all the positive identifications (i.e., samples containing an agent), while 1-specificity corresponds to the fraction of False Positive (FP) Identifications made out of all negative identifications (i.e., samples without an agent). These two characteristics are also referred to as TP and FP rates of a method.

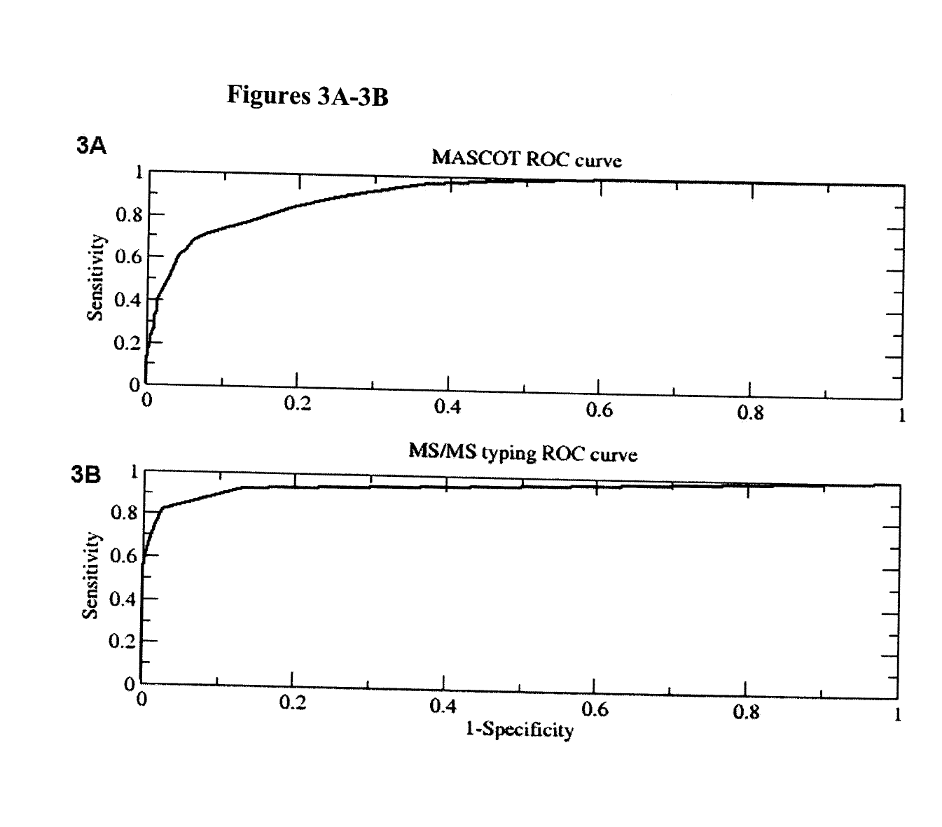

Using an ROC curve, the inventors determined that there are high correlation coefficients between two experimental spectra even with a significantly reduced amount of the compound of interest in one of the spectra, while the database searches fail to provide correct identification (See FIG. 3). Nearly double the increase in sensitivity of detection between database search results and MS/MS typing without loss of specificity is observed. Thus, the sensitivity of detection increases with the use of the experimental database reference library. The specificity of detection is similarly increased by addition of intensities (which are additional to fragment m/zs information) to the comparison.

The Pearson correlation coefficient ranges between −1 and 1, with 1 when a linear equation describes the relationship perfectly and positively, −1 shows that all data points lay on a single line but that Y increases as X decreases. In practical analysis, when correlation coefficient approaches 0, it can be interpreted as independence between the two variables, assuming they are normally distributed. If the comparison between a MS/MS spectrum measured in the sample to the species-specific MS/MS spectrum contained in the reference library shows a significant level of correlation (as suggested below), the organism which produced the reference library spectrum is considered detected in the sample.

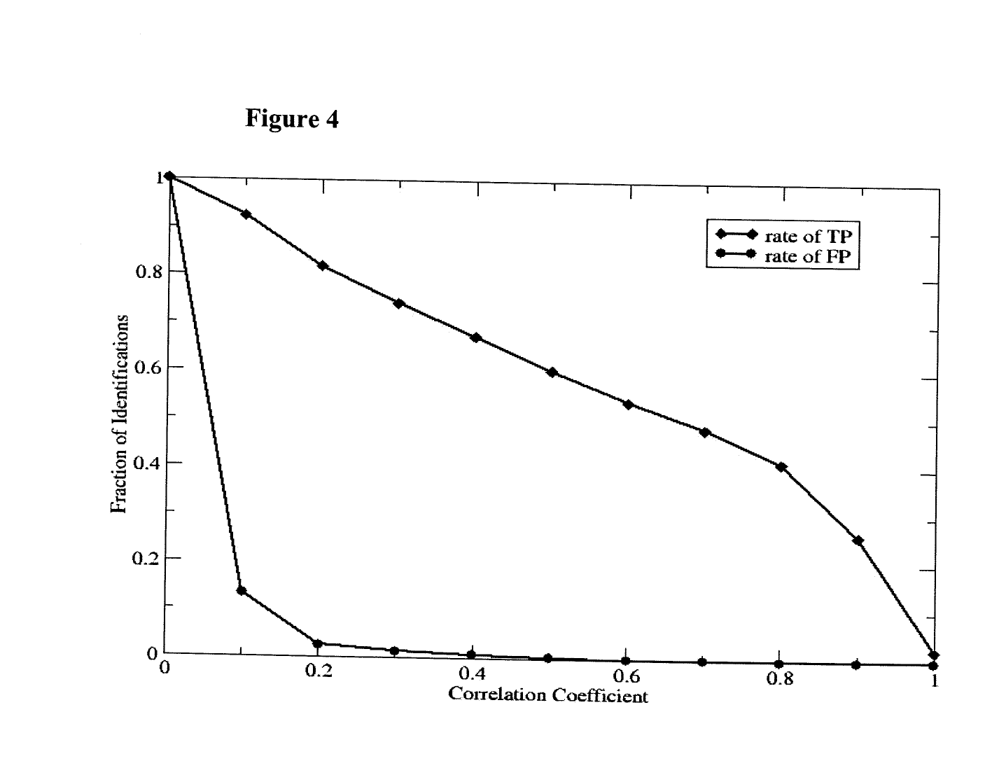

The significance of a correlation coefficient can either be assessed computationally by looking at a p-value, which reflects the probability of obtaining a particular correlation coefficient or higher by chance, or by statistical analysis of a dataset. To make a statistical assessment for significance of correlation coefficient, a plot TP and FP rates as a function of correlation coefficient has been made. The assessment for Pearson correlation method was performed using the same dataset of 1366 spectra as for the ROC curve comparison. The rates of TP and FP identifications as a function of Pearson correlation coefficient is shown in FIG. 4. It was found that in the dataset, at Pearson correlation coefficient cutoff of 0.5, one can detect 60% of all correct identifications, with 0.1% rate of false positives, while even at the correlation coefficient of 0.2, 82% of all correct identification can be detected with only 2% false positive rate.

### MS/MS Typing for Detection of Multiple Microorganisms.

MS/MS typing is a technique for detection of a single or multiple microorganisms in a single or multiple samples. Given a sample for analysis, MS/MS typing can detect a list of specified organisms or screen the sample for all the

The sample can be processed according to the sample processing procedures described above, which can either be specific for a specified microorganism (as described in the given example below) or involve a general procedure suitable for MS analysis. In case it is required to detect varied classes of microorganisms (such as bacteria, viruses, spores, toxins, etc), a set of different chemical procedures may be applied, such as for example splitting a single sample into several spots on the MALDI target plate processed and analyzed under different conditions (see U.S. patent Ser. No. 11/441,176, the entire contents of which are incorporated herein by reference).

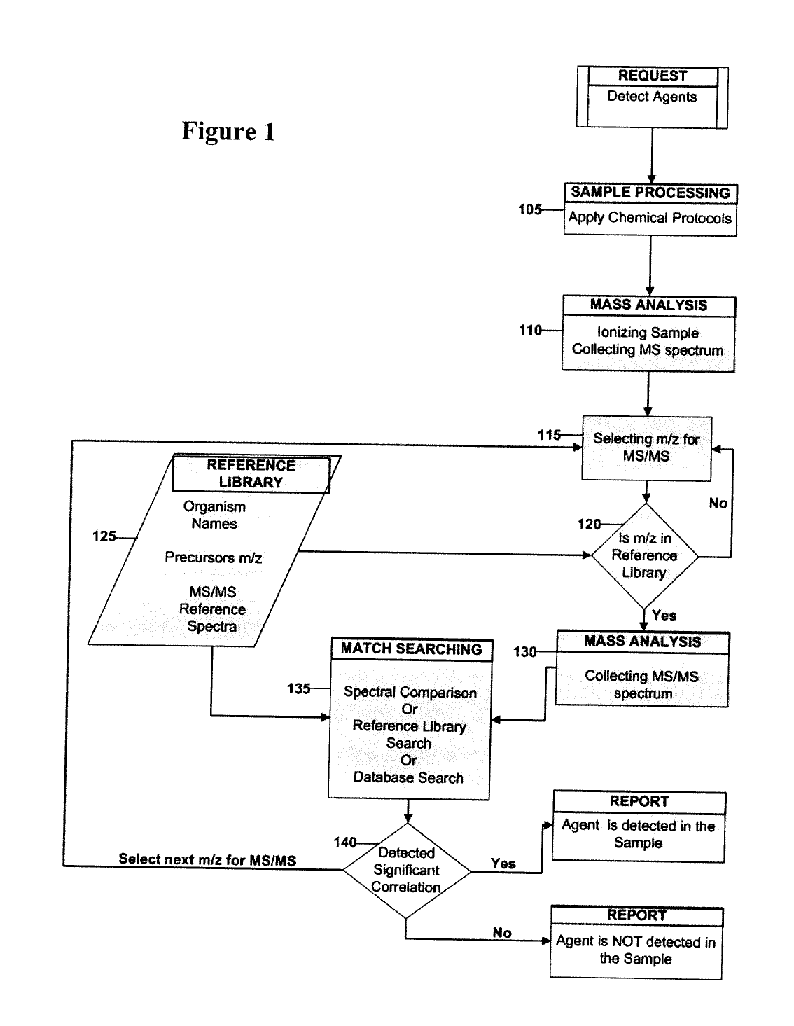

As illustrated in FIG. 1, to perform a requested organism identification(i.e., to perform a request to detect an agent), a chemically processed sample, as shown at step 105 (or several spots if different types of bio-agents are involved) will be subjected to mass spectrometry. The mass spectrometry measures and analyzes all (or most of) the peptide ions present in the sample, as shown by the mass analysis at step 110. A list of precursor m/z peaks arranged in the order of significance can be produced. The observed precursor m/z peaks can be cross-referenced against those in the reference library, as shown by the selecting m/z at step 115. The observed precursor m/z peaks corresponding to any m/z precursors from Reference Library can be MS/MS fragmented, each producing an MS/MS spectrum, as shown by the mass analysis at step 130. The resultant MS/MS spectrum can then be compared to the corresponding reference library spectrum, representing a particular bio-agent, using a spectral correlation comparison method, as shown at step 135 by the designation match searching.

If observed correlation coefficient (calculated between the two MS/MS spectra) is significant, i.e. exceeds the threshold which is determined by the user specified probability of false positive error, or significance level (typically, 2-5% range, corresponding to respective 98-95% confidence levels) the bio-agent can be reported as detected. The procedure may be repeated in a cycle between step 115 and step 140 for all of the produced MS/MS spectra (until there are no more significant precursor m/z peaks left) to detect multiple microorganisms in the sample or be performed for one specific precursor m/z if only one agent of interest is specified.

An exemplary general procedure for the detection of a single bio-agent is described in detail in Procedures 1-3 below.

### Procedure 1:

Sample Processing for Bacillus globigii.

Microcon YM-100 (100,000 MWCO) from Millipore Corporation filter (sample reservoir) was placed in a 1.5 mL microcentrifuge tube and 400 μL of sample was added. The sample was then centrifuged at 5000 rcf for 5 minutes using a benchtop centrifuge. The filter was then removed from the microcentrifuge tube and the contents in the microcentrifuge tube were discarded. The concentrated sample from the filter was then collected into a fresh microcentrifuge tube by placing the filter upside down in a fresh 1.5 mL microcentrifuge tube and centrifuging at 1000 rcf for 2 minutes. The final volume of the concentrated sample was adjusted to 10 μL and used for further analysis.

Examples and details regarding sample processing embodiment suitable for the present invention are described in N. I. Taranenko, B. Oktem, A. K. Sundaram, V. M. Doroshenko, Method and Apparatus for Processing of Biological Samples for Mass Spectrometry Analysis, in U.S. patent Ser. No. 11/441,176 filed May 26, 2006, the entire contents of which are incorporated herein by reference.

The small acid soluble proteins from B. globigii spores were extracted directly on to the C18-coated MALDI target probe surface at 50° C. and digested using immobilized trypsin. A probe clean up protocol was then used to remove impurities Details of probe clean techniques suitable for the present invention are described in the above-noted U.S. patent Ser. No. 11/441,175. Following a probe clan, a mass spectrum was recorded following digestion.

1 μL of the spore sample was placed on the target plate followed by 1 μL of 10% TFA and the resulting solution was allowed to dry out on the C18 probe surface. 1 μL of immobilized trypsin beads was then added to the dried out spot, followed immediately by 1 μL of acetonitrile. Digestion was allowed to occur until the spot dried out. Trypsin digested sample mixture was cleaned by washing with 3 μL of water (add 3 μL of water and remove it using pipette). 1 μL of matrix was then added on top of the washed spot (10 mg/mL solution of α-cyano-4-hydroxycinnamic acid matrix in 60% acetonitrile+0.1% TFA). This peptide-matrix mixture was allowed to dry and crystallize before recording the mass spectrum.

### Procedure 2:

Creating the Reference Library Spectrum for Bacillus globigii also Termed Bacillus subtilis:

Using the following sample processing (similar to that described in U.S. patent Ser. No. 11/441,176), B. globigii mass analysis was used to create a reference library spectrum entry for the microorganism. In this example, AP-MALDI MS and MS/MS spectra were then acquired using an LCQ Deca XP ion trap MS fitted with an AP-MALDI ionization source in positive ion mode. The MS spectrum was recorded for masses in a mass range under 2000 Da.

Two MS ions under m/z<2000 were reported to be present in B. globigii with m/z values of 1584.74 and 1927.93 (See Warscheid B and Fenselau C. Anal Chem. 2003 Oct. 15; 75(20):5618-27, the entire contents of which are incorporate herein by reference). Each of the mass ions were subjected to MS/MS analysis and submitted to MASCOT database search for identification. The mass ions were identified as corresponding peptides: LVSFAQQNMSGQQF and FEIASEFGVNLGAETTSR. These peptide sequences then were submitted for a full data search over NCBInr database (i.e., a non-redundant protein sequence database compiled by National Center for Biotechnology Information [NCBI]) to detect their species-specific uniqueness. It was found that LVSFAQQNMSGQQF is specific to B. globigii, while 1927.93 has a peptide sequence common to two organisms: Bacillus globigii and Bacillus licheniformis ATCC 14580. Therefore, only LVSFAQQNMSGQQF is considered a species specific biomarker to B. globigii, due to its uniqueness to one species.

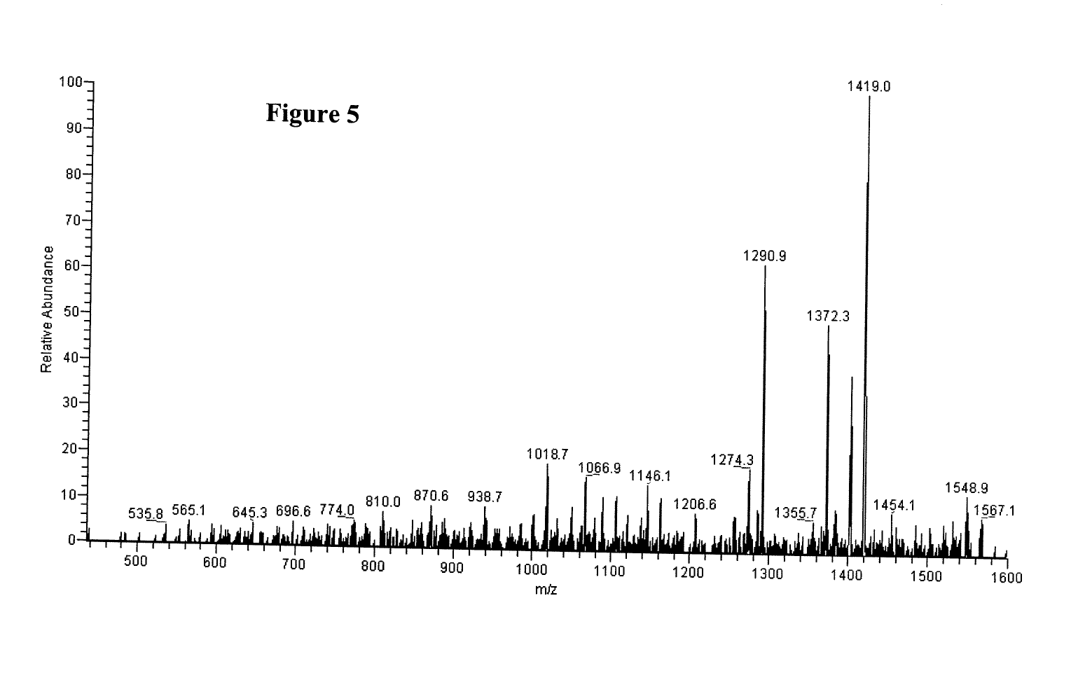

A number of MS/MS experiments were performed on the isolated m/z of 1584.74 with the concentrated sample of B. globigii to create a suitable MS/MS spectrum for the reference library. The best representative MS/MS spectrum of precursor with m/z=1584.74 after identification of LVSFAQQNMSGQQF peptide was saved in the reference library as the reference spectrum (see FIG. 5) for Bacillus globigii/Bacillus subtilis.

### Procedure 3:

MS/MS Typing for Detection of B. globigii.

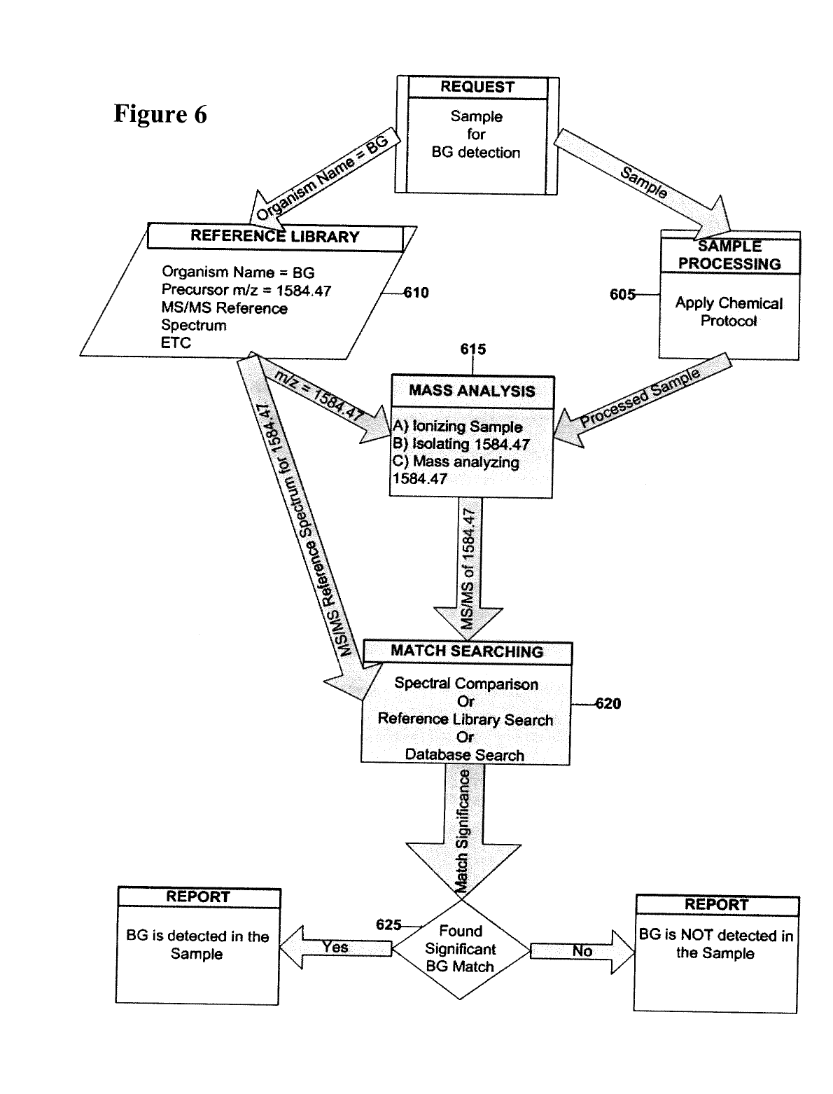

After creating the reference library entry for B. globigii, the following procedure was used in this example for detection of B. globigii in a biological sample as illustrated in FIG. 6. The sample is processed as using previously described sample processing protocol as shown in FIG. 6 at step 605, followed by mass analyzing the sample at step 615, which involves ionizing the sample, isolating specified precursor ion with m/z of 1584.47 (as is specified by the entry for B. globigii (BG) as stored in Reference Library shown at FIG. 6, step 610) and MS/MS fragmenting the precursor ion to produce an MS/MS spectrum at the precursor mass of B. globigii biomarker at step 615. The resultant MS/MS spectrum is then compared to the B. globigii reference spectrum contained in the Reference Library (FIG. 6, step 610) as shown in the FIG. 6 step 620, using one of the possible spectral comparison methods, for example Pearson correlation coefficient.

The organism is considered present in the sample when a significant correlation between the two spectra is observed as shown in FIG. 6, step 625. For example, in one instance of the experiment when the organism was present in the sample, the correlation coefficient between the precursor 1584.74 MS/MS spectrum and B. globigii reference spectrum showed correlation coefficient of 0.85, with probability of this value being obtained by chance (p-value) approaching 0, as is shown in the FIG. 4, the probability of getting a false positive identification with this correlation coefficient also approaches 0.

These examples serve as illustration as to the possible use of MS/MS typing technology without limiting its use.

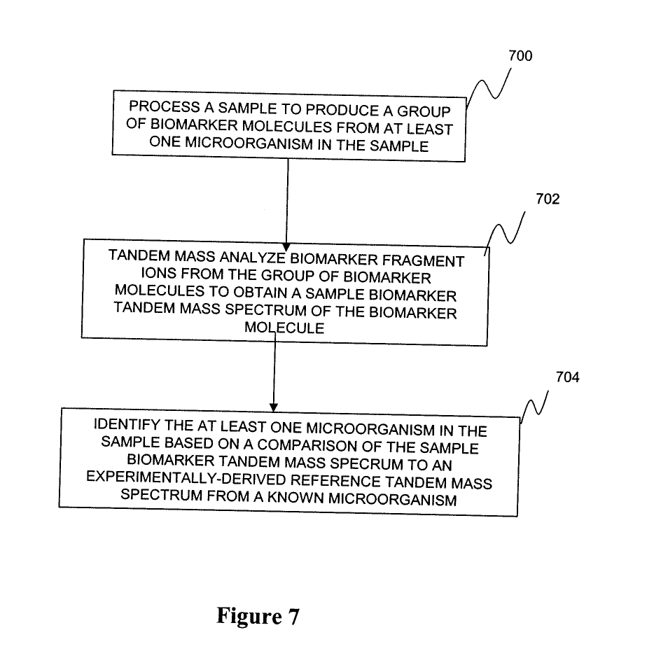

Processing Method: FIG. 7 is a flowchart depicting a method according to one embodiment of the present invention for identification of microorganisms in a sample based on identification of biomarkers originated from the microorganisms using mass spectrometry. As shown in FIG. 7, the method includes at 700 processing the sample to produce at least one group of biomarker molecules from at least one of microorganisms in the sample. At 702, the biomarker ions from the at least one group of biomarker molecules are mass-analyzed to obtain a sample biomarker mass (MS/MS) spectrum of the biomarker molecule. At 704, at least one of microorganisms in the sample are identified based on a comparison of the sample biomarker mass spectrum to an experimentally-derived reference mass spectrum from a known microorganism.

At 700, a sample of the microorganisms can be concentrated. The sample can be subjected to a solution including at least one of acid, base, or organic solvent. The sample can be digested into proteins for example by via proteolysis using trypsin or trypsin immobilized to a surface.

At 700, the biomarker molecules can be extracted from a membrane or cell volume of the microorganisms in the sample. The biomarker molecules can be produced by digesting proteins extracted from the membrane or cell volume of the microorganisms in the sample. The digesting can utilize trypsin and produce species-specific peptides as the biomarker molecules.

For example, an acid or a base solution can be applied to the microorganisms in the sample for extraction of the biomarker proteins. If for instance an acid solution is applied to an organic sample containing spores, the acid extracts a protein from the small acid-soluble protein (SASP) family, as the biomarker protein. Acid solutions such as for example trifluoroacetic acid (TFA) can be applied by the solution applicator for this and other extractions. Formic acid or acetic acid are applicable as well for this type of extraction as these acids are volatile and can be removed by evaporation subsequently. The extracted biomarker protein can be SAS2_BACSU (Mass: 7332 Small, acid-soluble spore protein 2 (SASP-2).—Bacillus subtilis) as given in Mass Spectrometry protein sequence Data Base (MSDB) database compiled by the Proteomics Group at Imperial College London.

In other examples, the microorganisms in the sample can include a virus, bacteria, a spore, a toxin, or a combination thereof, and ammonium hydroxide or tris-carbonate or a combination thereof can be applied as a base for extraction of the biomarker proteins. Tris buffer or NaOH are also applicable to extract this class of biomarkers such as 1AQ3A from MSDB database. (Mass: 13714 ms2 protein capsid mutant T59S, chain A—phage ms2).

A digesting medium capable of at least partial digestion of the biomarker proteins into peptides is applied. The digesting medium can include one or more enzymes such as trypsin, subtilisin, chymotrypsin, pepsin, papain, S. aureus V8, elastase, Lys-C endoproteinase, Arg-C endoproteinase, and Glu-C endoproteinase enzymes. The enzymes can be immobilized on tiny beads or surface to minimize autolysis.

The sample, the solutions, the digesting medium, and the biomarker proteins can be elevated to a temperature above 50° C., above 60° C., or above 67° C. 80° C. is considered a practical upper range suitable for many biological samples.

At 700, the sample can be cleaned up using at least one of: ZipTip, HPLC column, or washing out said sample in-situ, when analytes of interest are bound to a column or ZipTip, while the interfering materials are washed away. The analytes of interest are eluted from the column of ZipTip using appropriate solvent. In cleaning up the sample, solvents can be applied for dissolution of contaminants and then removed from the sample to remove at least a part of the contaminants. In certain cases, greater than 95% of contamination can be removed. Examples of contaminants removed include buffer salts, detergents, components of media used for growing cells, environmental or dust particles present in the bioaerosol collection, while retaining concentrations of proteins, peptides, lipids and toxins extracted from microorganisms or cells for analyte analysis. Suitable solvent applicators can be manual or automated dispensers including for example a solvent pipettor or dispenser. Suitable solvents include water, a volatile buffer like ammonium bicarbonate buffer, a non-volatile buffer such as tris-buffer, and phosphate buffered saline (PBS), organic solvents, ethanol, methanol, isopropanol, acetone, and/or acetonitrile.

At 702, the biomarker molecules can be ionized to obtain biomarker ions, and thereafter the biomarker ions are isolated and fragmented to obtain the biomarker fragment ion spectra. Various ionizing techniques are suitable for the present invention including for example matrix assisted laser desorption ionization (MALDI) operated at atmospheric pressure conditions or sub-atmospheric pressure or vacuum conditions. In MALDI, matrix materials such as for example α-cyano-4-hydroxycinnamic acid, 2,5-dihydroxybenzoic acid, sinapinic acid, ferulic acid, or a combination of the acids can be used.

Other ionizing techniques include using at least one of an infrared laser or an ultraviolet laser for laser ionization. Other ionizing techniques include Electrospray ionization (ESI). Various fragmentation processes are suitable for the present invention including fragmenting the biomarker ions using collision induced dissociation (CID) or fragmenting the biomarker ions using electron capture dissociation (ECD) or electron transfer dissociation (ETD). The biomarker ions in one embodiment can be fragmented using infra-red multiphoton dissociation (IRMPD) (See Little et al., Anal Chem. 1994, 66, 2809-2815, the entire contents of which are incorporated herein by reference). Alternatively, the biomarker ions can be fragmented using interactions with metastable molecules. The fragmented biomarker ions can be mass-analyzed using quadrupole ion trap mass spectrometer, quadrupole time-of-flight mass spectrometer, or Fourier transform ion cyclotron resonance mass spectrometer.

At 704, a search is made between the sample biomarker mass spectrum and the reference mass spectra to determine a match and that match is scored. Prior to the search, a library of reference MS/MS spectra of the biomarker molecules from known microorganisms can be created and stored in a library. The library can include reference MALDI-MS/MS spectra of biomarker molecules from known microorganisms, reference atmospheric pressure MALDI-MS/MS spectra of biomarker molecules from known microorganisms, reference ESI-MS/MS spectra of biomarker molecules from known microorganisms, experimental reference MS/MS spectra of the biomarker molecules from the known microorganisms, and/or theoretical reference MS/MS spectra of biomarker molecules from known microorganisms.

At 704, a search can be made between a sample biomarker tandem mass spectrum and a reference tandem mass spectrum having a similar precursor ion masses. A correlation coefficient can be calculated between a sample biomarker tandem mass spectrum and a reference tandem mass spectrum. The calculation can include calculating a fragment ion mass and an intensity correlation coefficient between the sample biomarker tandem mass spectrum and the reference tandem mass spectrum. The correlation coefficient can be scored as a metric of identification of the microorganism.

Scoring techniques can include the techniques discussed above and others such as for example Cosine similarity measure, Pearson correlation method, Spearman correlation, cross-correlation, regression analysis, χ²-method. A threshold for the correlation coefficient can be set and the microorganism identified based on a relation between the score and the threshold, as illustrated above.

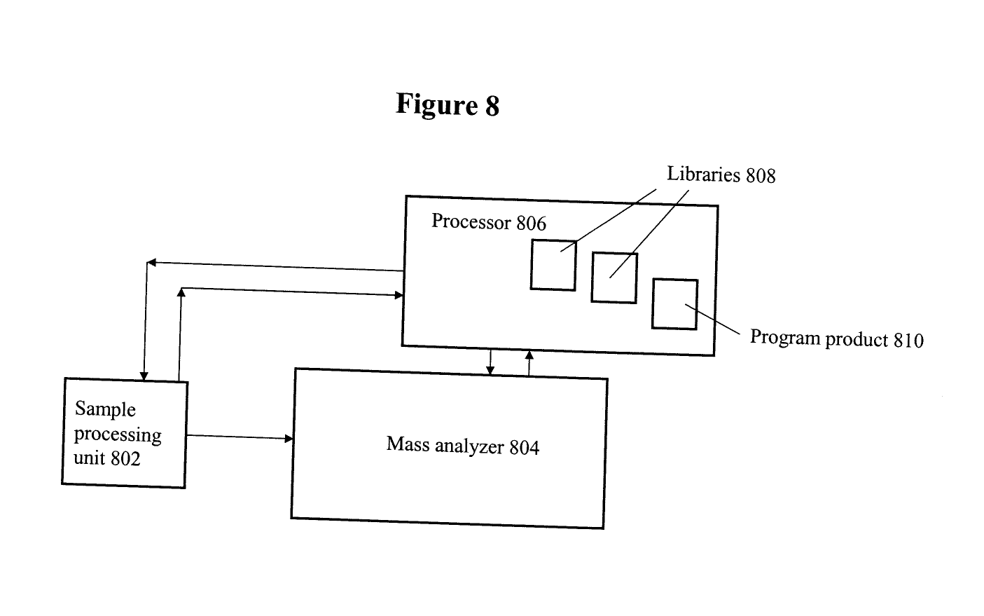

System Implementation: In one embodiment of the present invention, the above-described methods and equipment are included in a system for identification of microorganisms in a sample as shown in FIG. 8. The system 800 can include a sample processing unit 802 to process the sample and produce at least one group of biomarker molecules (e.g., molecules of a species-specific peptide) from at least one microorganism in the sample. The sample processing unit 802 can be configured to at least one of concentrate the sample, subject the sample to a solution including at least one of acid, base, or organic solvent, digest the sample into proteins via proteolysis, extract the biomarker molecules from a membrane or cell volume of the microorganisms in the sample, produce the biomarker molecules by digesting proteins extracted from a membrane or cell volume of the microorganisms in the sample, and remove at least some of background (interfering with detection) molecules from the sample.

Remaining molecules, including microorganism biomarkers are introduced into a mass-analyzer 804 which receives the at least one group of biomarker molecules from the sample processing unit and which mass analyzes biomarker ions and/or tandem mass analyzes biomarker fragment ions from the at least one group of biomarker molecules to obtain a sample biomarker tandem mass spectrum of the biomarker molecule. The mass-analyzer 804 can perform a number of processes independently or in concert. The mass-analyzer can ionize the at least one group of biomarker molecules to obtain biomarker ions and/or isolate and fragment the biomarker ions to obtain the biomarker fragment ions.

The mass analyzer 804 can ionize the biomarker molecules using at least one of matrix assisted laser desorption ionization (MALDI), using either infrared laser radiation or ultraviolet laser radiation or Electrospray ionization (ESI). The mass analyzer can perform ion dissociation to obtain biomarker fragment ions using at least one collision induced dissociation (CID), electron capture dissociation (ECD), electron transfer dissociation (ETD), multiphoton dissociation, and interactions with metastable molecules. The mass analyzer 804 can include at least one of a quadrupole ion trap mass spectrometer; a quadrupole time-of-flight mass spectrometer, and a Fourier transform ion cyclotron resonance mass spectrometer.

As shown in FIG. 8, a processor 806 taking data from the mass-analyzer 804 identifies the microorganism in the sample based on a comparison of the sample biomarker tandem mass spectrum to an experimentally-derived reference tandem mass spectrum from a known microorganism. The processor 806 is programmed to search for a match between the sample biomarker tandem mass spectrum and the reference tandem mass spectra and to score the match using any of the correlation techniques described above.

The processor 806 includes a number libraries 808 stored in memory therein or otherwise accessible to the processor 806. For example, the libraries 808 can be stored remotely and can be accessed by the processor 806 via a network. The libraries 808 include for example a library of reference tandem mass spectra (MS/MS) of the biomarker molecules from known microorganisms, a library of reference MALDI-based tandem mass spectra of biomarker molecules from known microorganisms, a library of reference atmospheric pressure MALDI-based tandem mass spectra of biomarker molecules from known microorganisms, a library of reference ESI-based tandem mass spectra of biomarker molecules from known microorganisms, a library of experimental reference tandem mass spectra of the biomarker molecules from the known microorganisms, and/or a library of theoretical reference tandem mass spectra of biomarker molecules from known microorganisms.

The processor 806 is programmed to perform a number of search and comparison routines. For example, the processor 806 can search between a sample biomarker tandem mass spectrum and a reference tandem mass spectrum having a similar precursor ion masses, calculate a correlation coefficient between a sample biomarker tandem mass spectrum and a reference tandem mass spectrum, calculate a fragment ion mass correlation coefficient between the sample biomarker tandem mass spectrum and the reference tandem mass spectrum, calculate a fragment ion mass and an intensity correlation coefficient between the sample biomarker tandem mass spectrum and the reference tandem mass spectrum, score the correlation coefficient as a metric of identification of the microorganism, and/or set a threshold for the correlation coefficient and identify the microorganism based on a relation between the score and the threshold.

The processor 806 may also be used to control mass analyzer 804 and/or sample processing unit 802. The processor 806 including appropriate software packages which permit control of mass analyzer 804 and/or sample processing unit 802.

Accordingly, in one embodiment of the present invention, the processor 806 may be implemented using a conventional general purpose computer or micro-processor programmed according to the teachings of the present invention, as will be apparent to those skilled in the computer art. Appropriate software can readily be prepared by programmers of ordinary skill based on the teachings of the present disclosure, as will be apparent to those skilled in the software art.

The processor 806 can be used to implement the method(s) of the present invention, wherein the computer of the processor 806 houses for example a motherboard containing a CPU, memory (e.g., DRAM, ROM, EPROM, EEPROM, SRAM, SDRAM, and Flash RAM), and other optical special purpose logic devices (e.g., ASICS) or configurable logic devices (e.g., GAL and reprogrammable FPGA). The computer also includes plural input devices, (e.g., keyboard and mouse), and a display card controlling a monitor. The computer can be used to drive any of the devices or to store any of the data or program codes listed in the appended claims such as for example the reference or sample mass spectrum, among others.

Additionally, the computer may include a floppy disk drive; other removable media devices (e.g. compact disc, tape, and removable magneto-optical media (not shown)); and a hard disk or other fixed high density media drives, connected via an appropriate device bus (e.g., a SCSI bus, an Enhanced IDE bus, or an Ultra DMA bus). The computer may also include a compact disc reader, a compact disc reader/writer unit, or a compact disc, which may be connected to the same device bus or to another device bus.

The computer of processor 806 can include at least one computer readable medium. Examples of computer readable media are compact discs, hard disks, floppy disks, tape, magneto-optical disks, PROMs (e.g., EPROM, EEPROM, Flash EPROM), DRAM, SRAM, SDRAM, etc. Stored on any one or on a combination of computer readable media, the present invention can include software for controlling both the hardware of the computer and for enabling the computer to interact with a human user or to interface and interact with the sample processing unit 802. Such software may include, but is not limited to, device drivers, operating systems and user applications, such as development tools.

Such computer readable media further includes the computer program product(s) or element(s) of the present invention for performing the inventive method(s) herein disclosed. The computer code devices of the present invention can be any interpreted or executable code mechanism, including but not limited to, scripts, interpreters, dynamic link libraries, Java classes, and complete executable programs. Moreover, parts of the processing of the present invention may be distributed for better performance, reliability, and/or cost.

In one embodiment of the present invention, there is provided a computer program product 810 encoded in a memory of processor 806 to identify a microorganism in a sample. The computer program product 810 includes a first computer program element programmed to identify the microorganism in the sample based on a comparison of a sample biomarker tandem mass spectrum taken from at least one group of biomarker molecules produced from the sample to an experimentally-derived reference tandem mass spectrum from a known microorganism. The computer program product 810 can also include a second computer program element programmed to receive an indication that the sample has been processed to produce the at least one group of biomarker molecules from the microorganism in the sample. The computer program product 810 can also include a third computer program element programmed to obtain the sample biomarker tandem mass spectrum taken from the at least one group of biomarker molecules.

The invention may also be implemented by the preparation of application specific integrated circuits or by interconnecting an appropriate network of conventional component circuits, as will be readily apparent to those skilled in the art.

Numerous modifications and variations on the present invention are possible in light of the above teachings. It is, therefore, to be understood that within the scope of the accompanying claims, the invention may be practiced otherwise than as specifically described herein.

1. A method for identification of microorganisms in a sample, comprising:
processing the sample to produce at least one group of biomarker molecules from at least one microorganism in the sample;
tandem mass-analyzing biomarker fragment ions from the at least one group of biomarker molecules to obtain a sample biomarker tandem mass spectrum of the biomarker molecule; and
identifying the at least one microorganism in the sample based on a comparison of the sample biomarker tandem mass spectrum to an experimentally-derived reference tandem mass spectrum from a known microorganism.

2. The method as in claim 1, wherein tandem mass-analyzing biomarker fragment ions comprises:
ionizing the at least one group of biomarker molecules to obtain biomarker ions; and
isolating and fragmenting said biomarker ions to obtain the biomarker fragment ions.

3. The method as in claim 1, wherein identifying at least one of microorganisms comprises:
searching for a match between said sample biomarker tandem mass spectrum and the reference tandem mass spectra and scoring said match.

4. The method as in claim 1, further comprising:
creating a library of reference tandem mass spectra (MS/MS) of known biomarker molecules from associated microorganisms.

5. The method as in claim 4, wherein creating said library comprises:
creating a library of reference MALDI-based tandem mass spectra of known biomarker molecules from associated microorganisms.

6. The method as in claim 4, wherein creating said library comprises:
creating a library of reference atmospheric pressure MALDI-based tandem mass spectra of known biomarker molecules from associated microorganisms.

7. The method as in claim 4, wherein creating said library comprises:
creating a library of reference ESI-based tandem mass spectra of known biomarker molecules from associated microorganisms.

8. The method as in claim 4, wherein creating said library comprises:
creating a library of experimental reference tandem mass spectra of known biomarker molecules from associated microorganisms.

9. The method as in claim 4, wherein creating said library comprises:
creating a library of theoretical reference tandem mass spectra of known biomarker molecules from associated microorganisms.

10. The method as in claim 1, wherein processing said sample comprises:
concentrating the sample.

11. The method as in claim 1, wherein processing said sample comprises:
subjecting the sample to a solution including at least one of acid, base, or organic solvent.

12. The method as in claim 1, wherein processing said sample comprises:
digesting the sample into peptides via proteolysis.

13. The method as in claim 12, wherein digesting said sample comprises:
digesting the sample into peptides with trypsin.

14. The method as in claim 13, wherein said digesting the sample into peptides comprises:
digesting the sample into peptides using trypsin immobilized to a surface.

15. The method as in claim 1, wherein processing said sample comprises:
extracting said at least one group of biomarker molecules from a membrane or cell volume of said at least one microorganism in the sample.

16. The method as in claim 1, wherein processing comprises:
producing said at least one group of biomarker molecules by digesting proteins extracted from a membrane or cell volume of said at least one microorganism in the sample.

17. The method as in claim 16, wherein said digesting proteins comprises digesting the proteins with trypsin.

18. The method as in claim 17, wherein producing said biomarker molecules comprises:
producing species-specific peptides as said at least one group of biomarker molecules.

19. The method as in claim 1, wherein processing said sample comprises:
removing at least some non-biomarker molecules from the sample.

20. The method as in claim 1, wherein tandem mass-analyzing biomarker fragment ions first comprises:
ionizing the at least one group of biomarker molecules using matrix assisted laser desorption ionization (MALDI).

21. The method as in claim 20, wherein tandem mass-analyzing biomarker fragment ions first comprises:
ionizing the at least one group of biomarker molecules under atmospheric pressure conditions.

22. The method as in claim 20, wherein tandem mass-analyzing biomarker fragment ions first comprises:
ionizing the at least one group of biomarker molecules by at least one of infrared laser radiation or ultraviolet laser radiation.

23. The method as in claim 1, wherein tandem mass-analyzing biomarker fragment ions first comprises:
ionizing the at least one group of biomarker molecules by electrospray ionization (ESI).

24. The method as in claim 1, wherein tandem mass-analyzing biomarker fragment ions comprises:
producing the biomarker fragment ions by collision induced dissociation (CID) of biomarker ions of the at least one group of the biomarker molecules.

25. The method as in claim 1, wherein tandem mass-analyzing biomarker fragment ions comprises:
producing the biomarker fragment ions by electron capture dissociation (ECD) of biomarker ions of the at least one group of the biomarker molecules.

26. The method as in claim 1, wherein tandem mass-analyzing biomarker fragment ions comprises:
producing the biomarker fragment ions by electron transfer dissociation (ETD) of biomarker ions of the at least one group of the biomarker molecules.

27. The method as in claim 1, wherein tandem mass-analyzing biomarker fragment ions comprises:
producing the biomarker fragment ions by multiphoton dissociation of biomarker ions of the at least one group of the biomarker molecules.

28. The method as in claim 1, wherein tandem mass-analyzing biomarker fragment ions comprises:
producing the biomarker fragment ions by interactions of metastable molecules with biomarker ions of the at least one group of the biomarker molecules.

29. The method as in claim 1, wherein tandem mass-analyzing biomarker fragment ions comprises:
mass-analyzing the biomarker fragment ions with a quadrupole ion trap mass spectrometer.

30. The method as in claim 1, wherein tandem mass-analyzing biomarker fragment ions comprises:
mass-analyzing the biomarker fragment ions with a quadrupole time-of-flight mass spectrometer.

31. The method as in claim 1, wherein tandem mass-analyzing biomarker fragment ions comprises:
mass-analyzing the biomarker fragment ions with a Fourier transform ion cyclotron resonance mass spectrometer.

32. The method as in claim 1, wherein identifying said microorganism comprises:
searching between a sample biomarker tandem mass spectrum and a reference tandem mass spectrum having a similar precursor ion masses.

33. The method as in claim 1, wherein identifying said microorganism comprises:
calculating a correlation coefficient between a sample biomarker tandem mass spectrum and a reference tandem mass spectrum.

34. The method as in claim 33, wherein calculating comprises:
calculating a fragment ion mass correlation coefficient between said sample biomarker tandem mass spectrum and the reference tandem mass spectrum.

35. The method as in claim 33, wherein calculating comprises:
calculating a fragment ion mass and an intensity correlation coefficient between said sample biomarker tandem mass spectrum and the reference tandem mass spectrum.

36. The method as in claim 33, further comprising:
scoring the correlation coefficient as a metric of identification of the microorganism.

37. The method as in claim 36, wherein scoring comprises:
scoring with at least one of: Cosine similarity measure, Pearson correlation method, Spearman correlation, cross-correlation, regression analysis, χ²-method.

38. The method as in claim 36, further comprising:
setting a threshold for said correlation coefficient and identifying the microorganism based on a relation between said score and said threshold.

39. A system for identification of microorganisms in a sample, comprising:
a sample processing unit configured to process the sample and produce at least one group of biomarker molecules from at least one microorganism in the sample;
a mass-analyzer configured to receive the at least one group of biomarker molecules from the sample processing unit and to tandem mass analyze biomarker fragment ions from the at least one group of biomarker molecules to obtain a sample biomarker tandem mass spectrum of the biomarker molecule; and
a processor configured to identify the at least one microorganism in the sample based on a comparison of the sample biomarker tandem mass spectrum to an experimentally-derived reference tandem mass spectrum from a known microorganism.

40. The system as in claim 39, wherein the mass-analyzer is configured to:
ionize the at least one group of biomarker molecules to obtain biomarker ions; and
isolate and fragment said biomarker ions to obtain the biomarker fragment ions.

41. The system as in claim 39, wherein the processor is configured to search for a match between said sample biomarker tandem mass spectrum and the reference tandem mass spectra and to score said match.

42. The system as in claim 39, wherein the processor comprises:
a library of reference tandem mass spectra (MS/MS) of known biomarker molecules from associated microorganisms.

43. The system as in claim 39, wherein the processor comprises:
a library of reference MALDI-based tandem mass spectra of known biomarker molecules from associated microorganisms.

44. The system as in claim 39, wherein the processor comprises:
a library of reference atmospheric pressure MALDI-based tandem mass spectra of known biomarker molecules from associated microorganisms.

45. The system as in claim 39, wherein the processor comprises:
a library of reference ESI-based tandem mass spectra of known biomarker molecules from associated microorganisms.

46. The system as in claim 39, wherein the processor comprises:
a library of experimental reference tandem mass spectra of known biomarker molecules from associated microorganisms.

47. The system as in claim 39, wherein the processor comprises:
a library of theoretical reference tandem mass spectra of known biomarker molecules from associated microorganisms.

48. The system as in claim 39, wherein the sample processing unit is configured to at least one of:
concentrate the sample;
subject the sample to a solution including at least one of acid, base, or organic solvent;
digest the sample into peptides via proteolysis;
extract said at least one group of biomarker molecules from a membrane or cell volume of said microorganisms in the sample;
produce said at least one group of biomarker molecules by digesting proteins extracted from a membrane or cell volume of said microorganisms in the sample; and
remove at least some non-biomarker molecules from the sample.

49. The system as in claim 39, wherein the mass analyzer is configured to ionize the at least one group of biomarker molecules by at least one of matrix assisted laser desorption ionization (MALDI) with infrared laser radiation or ultraviolet laser radiation, and electrospray ionization (ESI).

50. The system as in claim 39, wherein the mass analyzer is configured to dissociate biomarker ions of the at least one group of biomarker molecules by collision induced dissociation (CID), electron capture dissociation (ECD), electron transfer dissociation (ETD), and multiphoton dissociation, and interactions with metastable molecules.

51. The system as in claim 39, wherein the mass analyzer comprises at least one of a quadrupole ion trap mass spectrometer; a quadrupole time-of-flight mass spectrometer, and a Fourier transform ion cyclotron resonance mass spectrometer.

52. The system as in claim 39, wherein the processor is configured to at least one of:
search between a sample biomarker tandem mass spectrum and a reference tandem mass spectrum having a similar precursor ion masses;
calculate a correlation coefficient between a sample biomarker tandem mass spectrum and a reference tandem mass spectrum;
calculate a fragment ion mass correlation coefficient between said sample biomarker tandem mass spectrum and the reference tandem mass spectrum;
calculate a fragment ion mass and an intensity correlation coefficient between said sample biomarker tandem mass spectrum and the reference tandem mass spectrum;
score the correlation coefficient as a metric of identification of the microorganism; and
set a threshold for said correlation coefficient and identify the at least one microorganism based on a relation between said score and said threshold.

53. A computer program product encoded in a memory of a processor to identify a microorganism in a sample, said product comprising:
a first computer program element programmed to identify the microorganism in the sample based on a comparison of a sample biomarker tandem mass spectrum taken from at least one group of biomarker molecules produced from the sample to an experimentally-derived reference tandem mass spectrum from a known microorganism.

54. The product as in claim 53, further comprising:
a second computer program element programmed to receive an indication that the sample has been processed to produce said at least one group of biomarker molecules from the microorganism in the sample.

55. The product as in claim 54, further comprising:
a third computer program element programmed to obtain the sample biomarker tandem mass spectrum taken from the at least one group of biomarker molecules.
